(19) United States
(12) Patent Application Publication
(10) Pub. No.: US 2002/0016601 A1
(43) Pub. Date: Feb. 7, 2002

(54) Instruments and techniques for inducing neocollagenesis in skin treatments

(76) Inventor: John H. Shadduck, Tiburon, CA (US)

Correspondence Address: John H. Shadduck, 1490 Vistazo West, Tiburon, CA, 94920, US

(21) Appl. No.: 09/753,922

(22) Filed: Jan. 2, 2001

Related U.S. Application Data
(60) Provisional application No. 60/174,300, filed on Jan. 3, 2000.

(51) Int. Cl. A61B 17/50

(52) U.S. Cl. 606/131

(57) ABSTRACT

A system for removal of skin surface layers and rapid subsurface delivery of fluid therapeutic agents in a treatment to cause neocollagenesis in the dermis to reduce wrinkles and alter the architecture of the dermal layers. The hand-held device has a skin interface with an abrasive that engages and abrades the skin surface as it is translated across a treatment site. The skin interface defines a grooved or undulating surface structure that is intermediate to an arrangement of media inflow ports and the media outflow ports. A preferred working end has a central media outflow port that communicates with a remote negative pressure source via a flexible tube. The skin interface has a plurality of media inflow ports about its periphery. The skin interface is formed of a resilient material such as silicone to allow the working end to flex and atraumatically engage the skin surface as it is translated across a treatment site. In practicing a method of the invention, the system operator (i) actuates a negative pressurization source in fluid communication the media outflow port; (ii) translates the abrasive skin interface across a treatment site abrade a surfacemost layer; and (iii) thereby creates a negative pressure environment within subsurface layers to very rapidly draw the therapeutic fluid media into the subsurface layers though the skin surface that is denuded by the abrasive architecture of the skin interface.

### CROSS-REFERENCE TO RELATED APPLICATIONS

This application claims priority from Provisional U.S. Patent Application Ser. No. 60/174,300 (Docket No. S-ECI-010) filed Jan. 3, 2000 having the same title.

This application is also related to the following U.S. patent applications: Ser. No. 09/475,479 (Docket No. S-ECI-008) filed Dec. 30, 1999 titled Instruments and Techniques for Inducing Neocollagenesis in Skin Treatments; and Ser. No. 09/475,480 (Docket No. S-ECI-007) filed Dec. 30, 1999 titled Instruments and Techniques for Inducing Neocollagenesis in Skin Treatments. All of the above listed applications are incorporated herein in their entirety by these references.

### BACKGROUND OF THE INVENTION

1. Field of the Invention

The present invention relates to devices for dermatology and more particularly to a skin treatment system that utilizes a skin interface with a spaced apart configuration of media outlow ports and media inflow port coupled to a negative pressure source and a therapeutic fluid source, respectively, for the very rapid subsurface delivery of therapeutic fluids to subsurface skin surface layers as the skin interface is translated over a patient's skin.

2. Description of Background Art

Dermatologists and plastic surgeons have used various methods for removing superficial skin layers to cause the growth of new skin layers (i.e., commonly described as skin resurfacing techniques) since the early 1900's. Early skin resurfacing treatments used an acid such as phenol to etch away surface layers of a patient's skin that exhibited damage which thereafter were replaced by new skin. (The term damage when referring to a skin disorder is herein defined as any cutaneous defect, e.g., including but not limited to rhytides, hyperpigmentation, acne scars, solar elastosis, other dyschromias, stria distensae, seborrheic dermatitus).

Following the removal of surface skin layers at a particular depth, no matter the method of skin removal, the body's natural wound-healing response begins to regenerate the epidermis and underlying wounded skin layers. The new skin layer will then cytologically and architecturally resemble a younger and more normal skin. The range of resurfacing treatments can be divided generally into three categories based on the depth of the skin removal and wound: (i) superficial exfoliations or peels extending into the epidermis, (ii) medium-depth resurfacing treatments extending into the papillary dermis, and (iii) deep resurfacing treatments that remove tissue to the depth of the reticular dermis (see FIGS. 1A-1B).

Modern techniques for skin layer removal include: CO₂ laser resurfacing which falls into the category of a deep resurfacing treatment, Erbium laser resurfacing which generally is considered a medium-depth treatment; mechanical dermabrasion using high-speed abrasive wheels which results in a medium-depth or deep resurfacing treatment; and chemical peels which may range from a superficial to a deep resurfacing treatment, depending on the treatment parameters. A recent treatment, generally called micro-dermabrasion, has been developed that uses an air-pressure source to deliver abrasive particles directly against a patient's skin at high-velocities to abrade away skin layers. Such a micro-dermabrasion modality may be likened to sand-blasting albeit at velocities that do not cause excessive pain and discomfort to the patient. Micro-dermabrasion as currently practiced falls into the category of a superficial resurfacing treatment.

A superficial exfoliation, peel or abrasion removes some or all of the epidermis (see FIGS. 1A-1B) and thus is suited for treating very light rhytides. Such a superficial exfoliation is not effective in treating many forms of damage to skin. A medium-depth resurfacing treatment that extends into the papillary dermis (see FIG. 1B) can treat many types of damage to skin. Deep resurfacing treatments, such as CO₂ laser treatments, that extend well into the reticular dermis (see FIG. 1B) cause the most significant growth of new skin layers but carry the risk of scarring unless carefully controlled.

It is useful to briefly explain the body's mechanism of actually resurfacing skin in response to the removal of a significant depth of dermal layers. Each of the above-listed depths of treatment disrupts the epidermal barrier, or a deeper dermal barrier (papillary or reticular), which initiates varied levels of the body's wound-healing response. A superficial skin layer removal typically causes a limited wound-healing response, including a transient inflammatory response and limited collagen synthesis within the dermis. In a medium-depth or a deep treatment, the initial inflammatory stage leads to hemostasis through an activated coagulation cascade. Chemotactic factors and fibrin lysis products cause neutrophils and monocytes to appear at the site of the wound. The neutrophils sterilize the wound site and the monocytes convert to macrophages and elaborate growth factors which initiate the next phase of the body's wound-healing response involving granular tissue formation. In this phase, fibroblasts generate a new extracellular matrix, particularly in the papillary and reticular dermis, which is sustained by angiogenesis and protected anteriorly by the reforming epithelial layer. The new extracellular matrix is largely composed of collagen fibers (particularly Types I and Ill) that are laid down in compact parallel arrays (see FIG. 1B). It is largely the collagen fibers that provide the structural integrity of the new skin—and contribute to the appearance of youthful skin.

All of the prevalent types of skin damage (rhytides, solar elastosis effects, hyperpigmentation, acne scars, dyschromias, melasma, stria distensae) manifest common histologic and ultrastructural characteristics, which in particular include disorganized and thinner collagen aggregates, abnormalities in elastic fibers, and abnormal fibroblasts, melanocytes and keratinocytes that disrupt the normal architecture of the dermal layers. It is well recognized that there will be a clinical improvement in the condition and appearance of a patient's skin when a more normal architecture is regenerated by the body's wound-healing response. Of most significance to a clinical improvement is skin is the creation of more dense parallel collagen aggregates with decreased periodicity (spacing between fibrils). The body's wound-healing response is responsible for synthesis of these collagen aggregates. In addition to the body's natural wound healing response, adjunct pharmaceutical treatments that are administered concurrent with, or following, a skin exfoliations can enhance the development of collagen aggregates to provide a more normal dermal architecture in the skin—the result being a more youthful appearing skin.

The deeper skin resurfacing treatments, such as laser ablation, chemical peels and mechanical dermabrasion have drawbacks. The treatments are best used for treatments of a patient's face and may not be suited for treating other portions of a patient's body. For example, laser resurfacing of a patient's neck or décolletage may result in post-treatment pigmentation disorders. All the deep resurfacing treatments are expensive, require anesthetics, and must be performed in a clinical setting The most significant disadvantage associated with deep resurfacing treatments relates to the post-treatment recovery period. It may require up to several weeks or even months to fully recover and to allow the skin the form a new epidermal layer. During a period ranging from a few weeks to several weeks after a deep resurfacing treatment, the patient typically must wear heavy make-up to cover redness thus making the treatment acceptable only to women.

The superficial treatment offered by micro-dermabrasion has the advantage of being performed without anesthetics and requiring no extended post-treatment recovery period. However, micro-dermabrasion as currently practices also has several disadvantages. First, a micro-dermabrasion treatment is adapted only for a superficial exfoliation of a patient's epidermis which does not treat many forms of damage to skin. Further, the current micro-dermabrasion devices cause abrasive effects in a focused area of the skin that is very small, for example a few mm.², since all current devices use a single pin-hole orifice that jets air and abrasives to strike the skin in a highly focused area. Such a focused treatment area is suitable for superficial exfoliations when the working end of the device is passed over the skin in overlapping paths. Further, such focused energy delivery is not well suited for deeper skin removal where repeated passes may be necessary. Still further, current micro-dermabrasion devices are not suited for deeper skin removal due to the pain associated with deep abrasions. Other disadvantages of the current micro-dermabrasion devices relate to the aluminum oxide abrasive particles that are typically used Aluminum oxide can contaminate the working environment and create a health hazard for operators and patients alike. Inhalation of aluminum oxide particles over time can result in serious respiratory disorders.

### SUMMARY OF THE INVENTION

The present invention comprises a hand-held instrument adapted for rapid delivery of therapeutic agents to subsurface skin layers in a periodic treatment to induce neocollagenesis in the dermis to reduce wrinkles and alter the architecture of the dermal layers. One embodiment of the system comprises a hand-held instrument with a resilient working skin interface (i) that carries a diamond abrasive treatment region in an undulating surface structure for abrading the skin surface in a controlled manner; (ii) a therapeutic fluid source coupled to media inflow ports about a periphery of the skin interface; and (iii) a negative pressure source coupled to a media outflow port in the center of the skin interface for drawing the therapeutic fluid into subsurface skin layers as well as for carrying skin debris from a treatment site.

More in particular, the working end that carries the resilient skin interface and sharp-edged diamond fragments is detachable from an intermediate instrument body and is inexpensive and disposable after being used for a single treatment. A preferred embodiment of hand-held instrument has a cartridge-type fluid reservoir that is detachable from the intermediate body section and is disposable. The working end defines a skin interface that engages and abrades the skin as it is translated across a treatment site. A preferred working end has a central aspiration channel and media outflow port that communicate with a remote negative pressure source via a flexible tube. The skin interface is configured with a plurality of fluid media inflow ports generally about a periphery of the skin interface. Of particular interest, a grooved or undulating surface structure is provided intermediate to the arrangement of media inflow ports and the media outflow port(s). The skin interface is formed of a resilient material such as silicone to allow the working end to flex and atraumatically engage the skin surface as it is translated across a treatment site.

The combination of the negative pressure (aspiration) source and the undulating groove structure of the system serves multiple functions: (i) to engage the skin surface within the skin interface by suction forces to allow the abrasive architecture to perform a method of removing surface layers as it is translated over the skin; (ii) to create a negative pressure condition within subsurface tissue layers to draw the therapeutic fluid media into the subsurface layers from the surface; and (iii) to aspirate the skin debris from the skin surface to a remote collection reservoir with a portion of the fluid media that moves across the skin surface.

In practicing a method of the invention, the following steps are performed, the system operator (i) places the skin interface carrying the abrasive architecture against the patient's skin in a treatment site; (ii) actuates a negative pressurization source in fluid communication the media outflow port(s); (iii) translates the abrasive skin interface across a treatment site abrade a surfacemost layer; and (iv) thereby creates a negative pressure environment within subsurface layers to rapidly draw the therapeutic fluid media into the subsurface layers though the partly denuded skin surface.

In general, the invention provides a system and techniques for rapidly delivering therapeutic fluid media to subsurface skin layers in a controlled manner.

The invention advantageously provides a technique to induce neocollageneis in a patient's skin to reduce wrinkles.

The invention advantageously provides a system that rapidly delivers a therapeutic fluid agent to subsurface skin layers such as an antibiotic agent, an anti-inflammatory agent, an anesthetic agent or a growth factors for inducing collagen synthesis.

The invention advantageously provides a system with a resilient skin interface that flexes to conform to the skin surface as it is translated across a skin treatment site.

The invention advantageously provides a fluid source for supplying a fluid to a perimeter of the skin interface to cool the skin, hydrate the skin and remove skin debris from the skin interface during use.

The invention advantageously provides an instrument handle with a cartridge-type fluid reservoir that is removable and disposable.

Other objects and advantages of the invention will be apparent from the following description, the accompanying drawings and the appended claims. Additional advantages and features of the invention appear in the following description in which several embodiments are set forth.

### BRIEF DESCRIPTION OF THE DRAWINGS

FIGS. 1A-1B are sectional illustrations of a patient's skin showing dermal layers.

FIG. 2A is a perspective view of an exemplary Type “A” body and working end of the instrument of the invention.

FIG. 2B is an exploded perspective view of components of the exemplary body and working end of FIG. 2A.

FIG. 3 is an enlarged perspective view of the working end of the instrument of FIGS. 2A-2B.

FIG. 4 is a sectional view of the exemplary working end of FIG. 3 taken along line 4-4 of FIG. 3.

FIG. 5 is a greatly enlarged sectional view of a skin interface portion of the exemplary working end of FIG. 3 showing diamond particles carried therein taken along line 5-5 of FIG. 4.

FIG. 6 is a sectional view of an alternative working end that is similar to the working end of FIG. 3.

FIG. 7 is a sectional view of another alternative working end that is similar to the working end of FIG. 3.

FIG. 8A is a greatly enlarged sectional view of a working end surface shown engaging a patient's skin in a method of the invention.

FIG. 8B is a sectional view of the working end surface of FIG. 9A showing a radial groove.

FIG. 9 is a perspective view of an alternative working end similar to that of FIG. 3 with an alternative plan shape.

FIGS. 10A-10B are perspective and a sectional views of a Type “B” system adapted for ultra-rapid delivery of therapeutic agents to subsurface tissue layers.

FIG. 11 is a sectional view of the working end surface of FIG. 10B shown engaging a patient's skin and rapidly delivering a fluid therapeutic agents to a targeted subsurface tissue layer in a method of the invention.

FIG. 12 is a sectional view of a skin interface shown engaging a patient's skin and causing a negative pressure gradient in subsurface dermal layers to rapidly migrate extracellular fluids and growth factors to more anterior dermis from the less anterior dermis to enhance neocollagenesis.

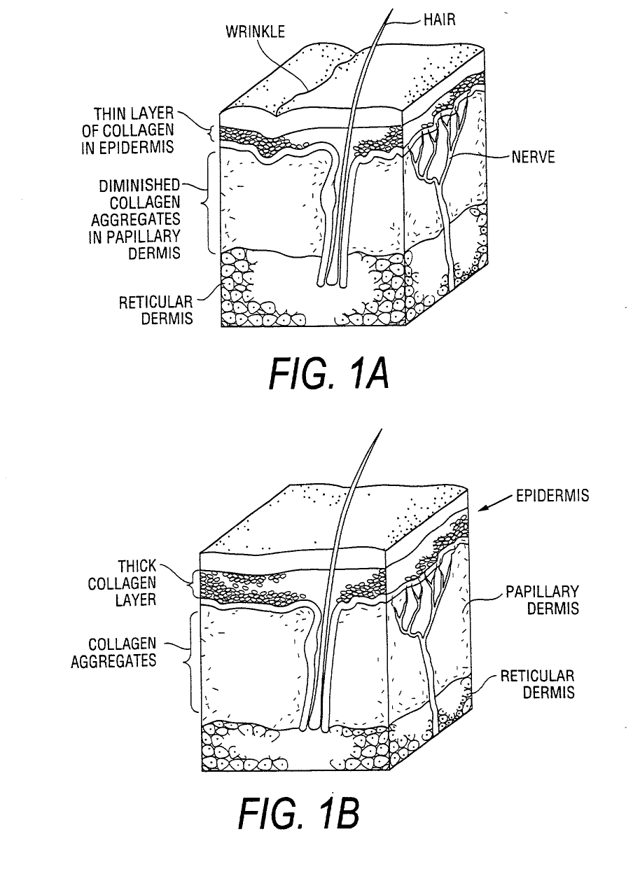

### DETAILED DESCRIPTION OF THE INVENTION

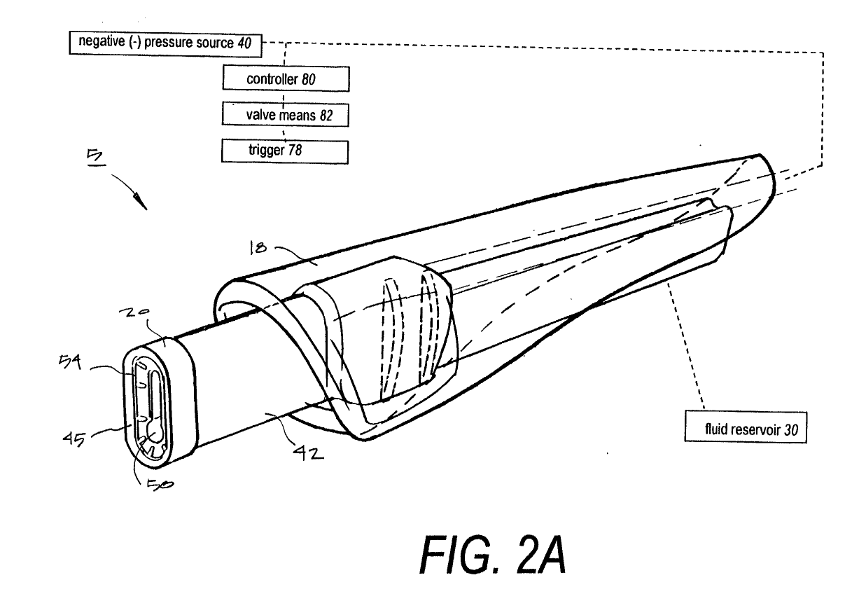

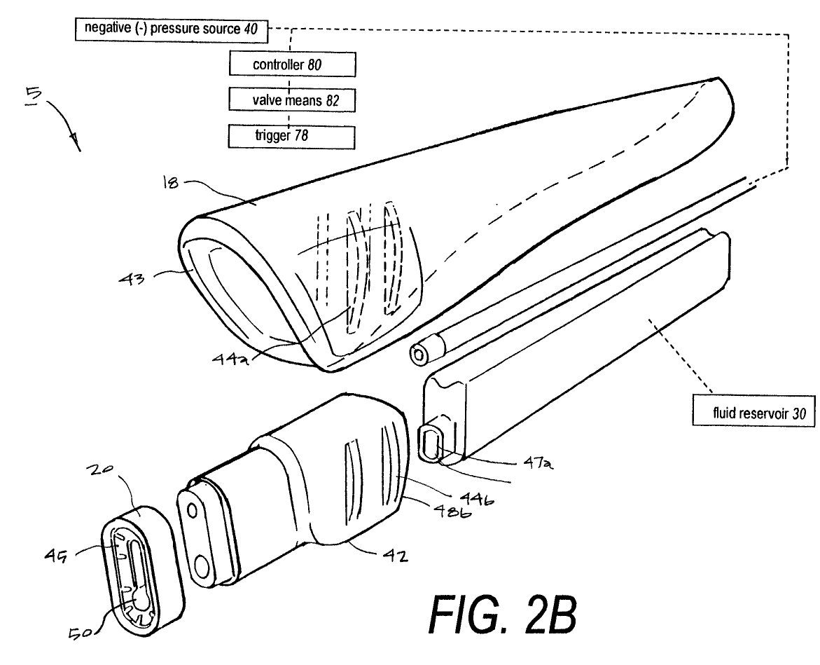

1. Type “A” Skin Treatment System. FIGS. 2A-2B illustrate an exemplary instrument system 5 that is designed for atraumatic removal of skin layers in a skin resurfacing or rejuvenation treatment. This embodiment of instrument system 5 comprises (i) a hand-held instrument 18 with a removable working end portion 20; (ii) a fluid source or reservoir 30 for supplying fluids to the working end 20; and (iii) a negative (−) pressure source (or aspiration source) indicated at 40 that aspirates fluid and skin debris from a treatment site TS on the patient's skin.

FIG. 2A shows that the working end 20 is carried by, and preferably but optionally detachable from, an intermediate body section indicated at 42. This embodiment has fluid reservoir indicated at 30 that also is detachably coupled to body section 42. This instrument body 18 is adapted to use a detachable ergonomic handle portion 43 that has mating ribs 44a that cooperate with notches 44b in body section 42. In this preferred embodiment, the reservoir 30 carrying a fluid F is a disposable cartridge that may be fitted to body section 42 with a breakable seal 46 and male and female fittings 47a and 47b as are known in the art (see FIG. 2B). The working end 20 described below also is detachable, inexpensive and disposable.

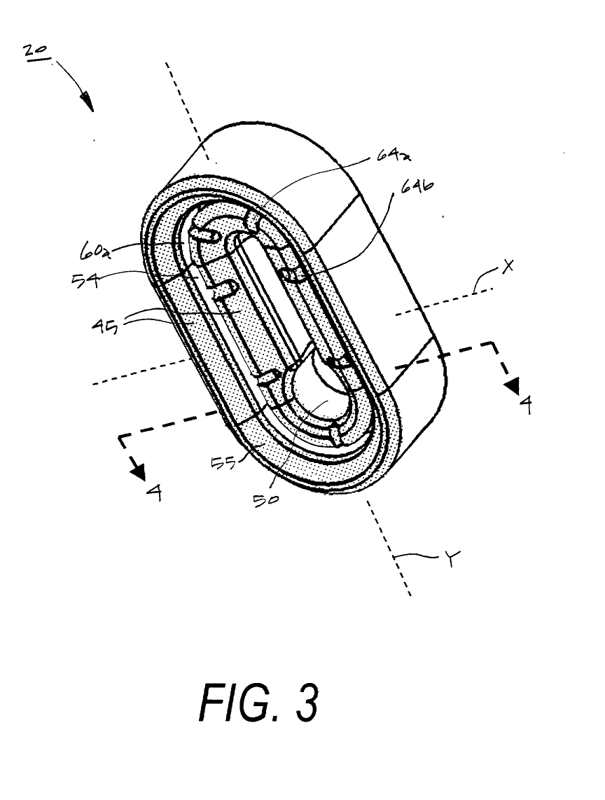

More in particular, referring to FIG. 3, the working end 20 defines a skin interface surface portion indicated at 45 that is adapted to engage the skin surface as the working end is translated across a patient's skin. In this embodiment, the skin interface 45 comprises the distal-facing surface region of the working end that is within the concave form 48 of working end 20. In this exemplary embodiment, the skin interface 45 transitions into an opening portion 50 generally centrally located in the working end. The opening 50 communicates with an interior passageway 51 that extends through body section 42 and further communicate with flexible tubing 52 that extends to the remote negative (−) pressure source 40 (see FIG. 2B). In this embodiment, there are a plurality of inflow apertures 54 generally about a perimeter of the skin interface that are in fluid communication with the fluid reservoir 30 as will be described further below.

Of particular interest, referring to FIG. 3, the skin interface 45 and optionally the entire working end 20 is formed of any suitable resilient material to allow the working end and skin interface 45 to flex and bend to atraumatically engage the skin surface as the working end is translated across a treatment site. It has been found that silicone is an ideal material that flexes desirably as described below in a method of the invention.

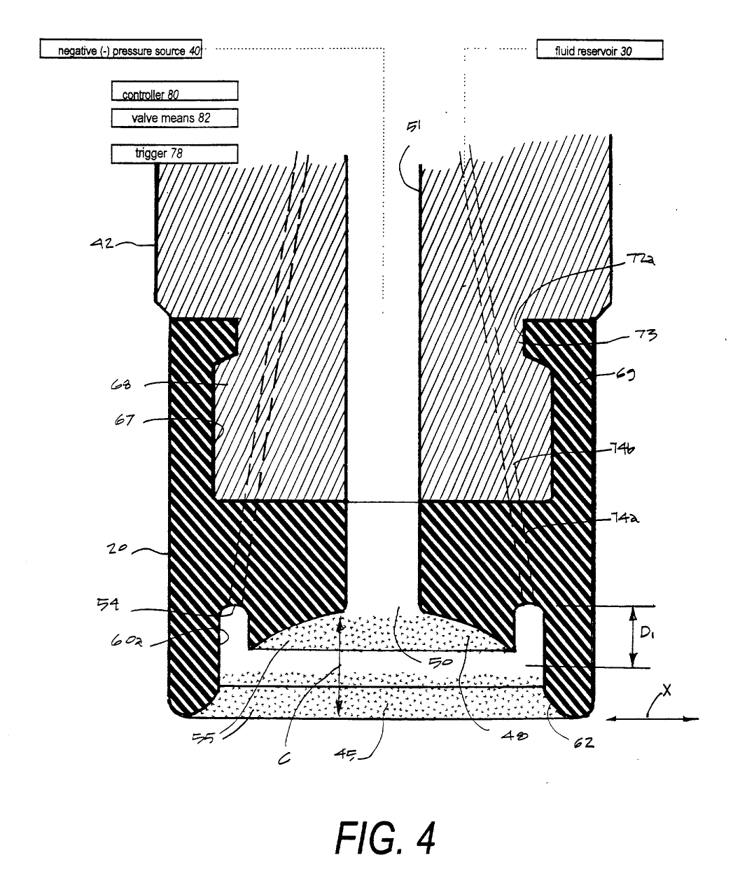

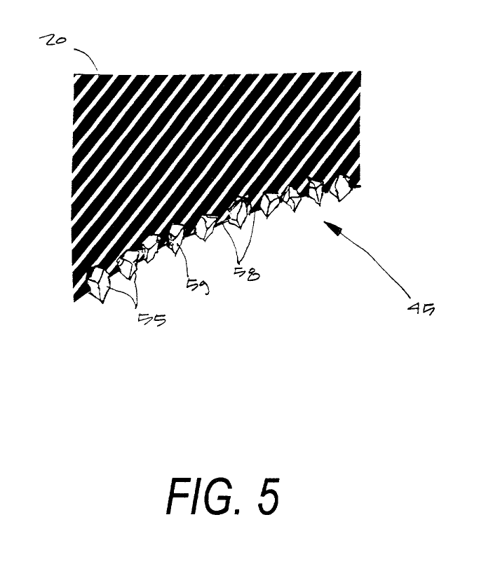

Also of particular interest to the invention, referring to FIGS. 4 and 5, the skin interface 45 carries a diamond dust, fragment or particulate composition indicated at 55. A natural diamond fragrant composition 55 has been found to have very sharp projecting points, edges and apices 57 that create an abrasive architecture that is well adapted to remove skin surface layers as the interface 45 carrying the diamond fragments is moved across a treatment site. Further, it has been found that spaces indicated at 58 between the diamond apices 57 will not tend to collect skin detritus when a fluid F (e.g., sterile water) generally flows across and about the skin interface 45 under the force of negative pressure from the aspiration source 40. It is believed that such removed skin particles or skin detritus do not adhere about the abrasive architecture of the skin interface 45 due to the fact that (i) the facets 59 of the diamond particles are very smooth and resist tissue adherence, and (ii) that the resilient material carrying the diamond fragments (e.g., silicone) is naturally lubricious and non-stick thus resisting any clogging of the spaces in the architecture of the skin interface 45. The ability of the fluid flow across the skin interface to remove skin detritus to the interior opening 50 for aspiration to a collection reservoir is very important for performing the method of the invention. As will be described below, the working end is passed over patient's skin in numerous paths, and the skin interface 45 must be continually free of detritus to allow a predictable level of skin surface removal during each portion of the working end's translation over a treatment site. It has been found that diamond fragments ranging in size from about 10 μm to about 250 μm in maximum cross-sectional dimension may be used in the skin interface to remove tissue. The different sizes of fragments cause very different characteristics in skin surface removal, and it has been found that different skin types and different desired depths of skin surface removal can be optimized by using a selected size of diamond fragment. For a thin, sensitive skin and a thin layer removal, preferably, the diamond crystals are from about 10 μm to about 50 μm in maximum cross-sectional dimension. For a thicker skin, or oily skin, and for a deeper layer removal, preferably, the diamond crystals are from about 30 μm to about 100 μm in maximum cross-sectional dimension.

Since each range of dimensions of the diamond fragments produces a differing ability to cut skin surface layers, another feature of the invention is to provide color coding to different working ends 20 that carry different dimensions of diamond fragments. It is believed that three to six colors may be appropriate for different ranges of cutting ability. In this embodiment, the molded silicone of the skin interface can be colored. A preferred method has been developed for partially embedding the diamond fragments 55 in the skin interface which comprises distributing a very thin, dispersed layer of the fragments in an injection mold and thereafter introducing silicone into the mold. By using a selected viscosity of introduced silicone and a selected dispersion of the fragments in the mold, the silicone will form and set about the fragments 55 as generally indicated in FIG. 5.

As shown in FIGS. 34, in this embodiment, the skin interface 45 defines an x-axis (X) and a y-axis (Y) wherein the direction of movement of the working end across a treatment site is generally in a direction along the x-axis as the operator sweeps the skin interface over the treatment site. The skin interface 45 has an overall transverse or x-axis dimension is from about 5.0 mm. to about 40.0 mm. with a larger dimensioned end being adapted for treating a larger skin area (e.g., arms, back, legs and décolletage). A typical x-axis dimension is from about 5.0 mm. to 15.0 mm. for a skin treatment site area TS around a patient's face. The dimension across the y-axis of the skin interface 45 may also be from about 5.0 mm. to about 40.0 mm. with the relation between the y-axis and x-axis being from 1:1 to about 3:1. In a preferred embodiment, the ratio y-axis/x-axis ratio is from about 1:1 (as is a round working end) to about 2:1 as shown in the plan shape of FIG. 3 or in an oval plan shape. The surface area of the skin interface 45 (e.g., in mm.²) about opening 50 may be from about 5.0 mm.² to about 100.0 mm.² to remove skin surface layers efficiently. It has been found that the shape of the concave form 48 of the working end is very important for the practice of the method and the depth C of the concavity may range from about 0.5 mm. to about 10.0 mm. depending on the overall dimensions across the working end (see FIG. 4).

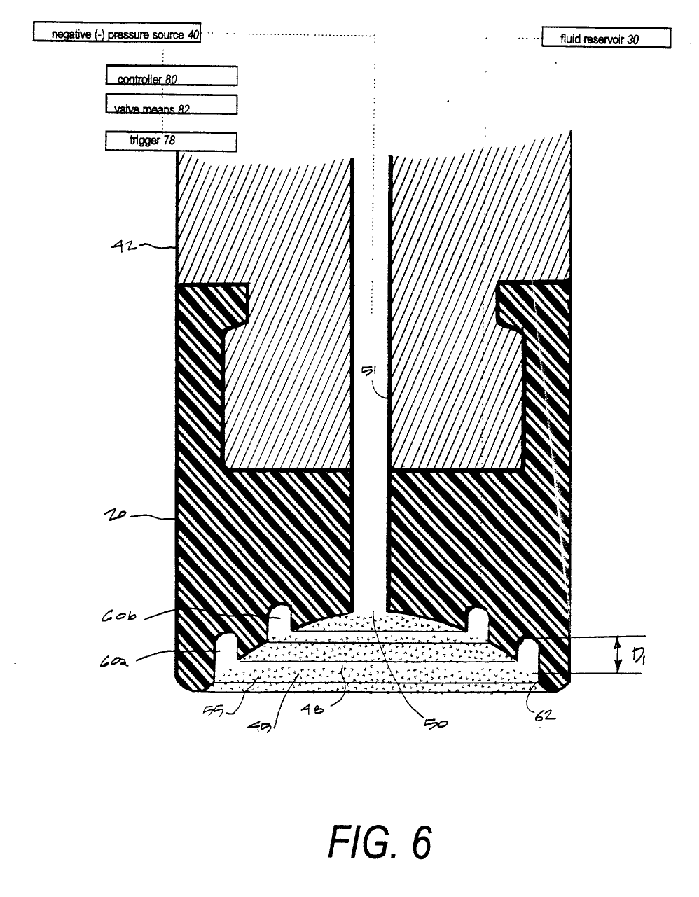

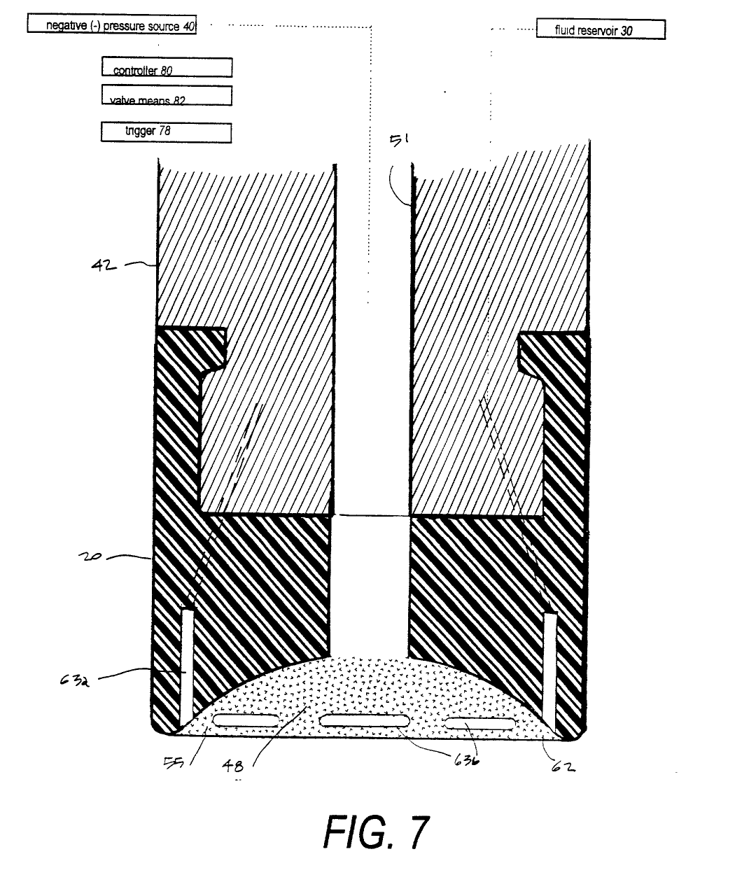

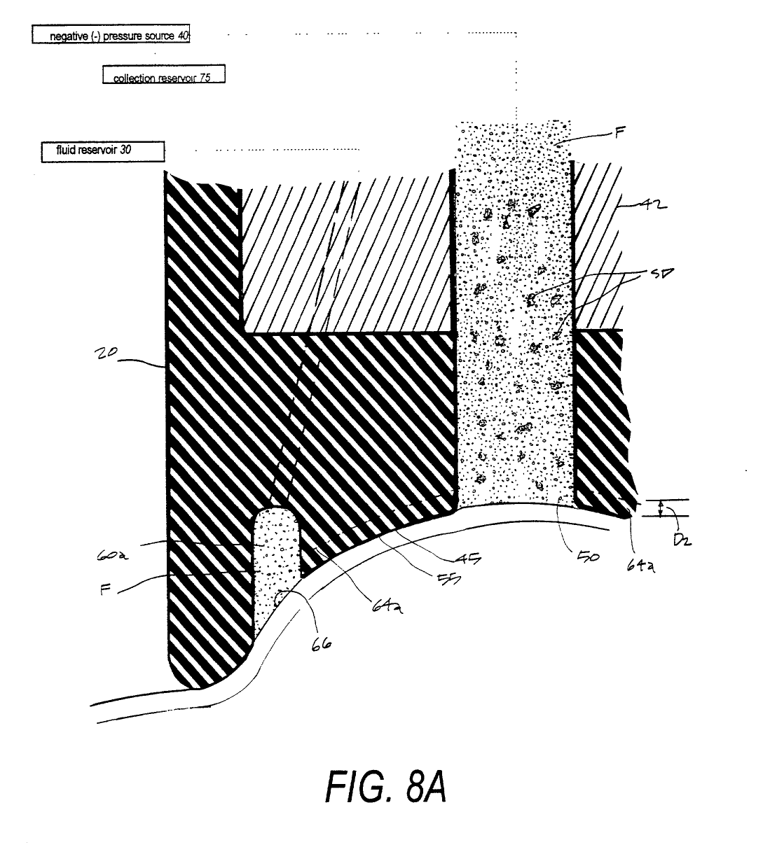

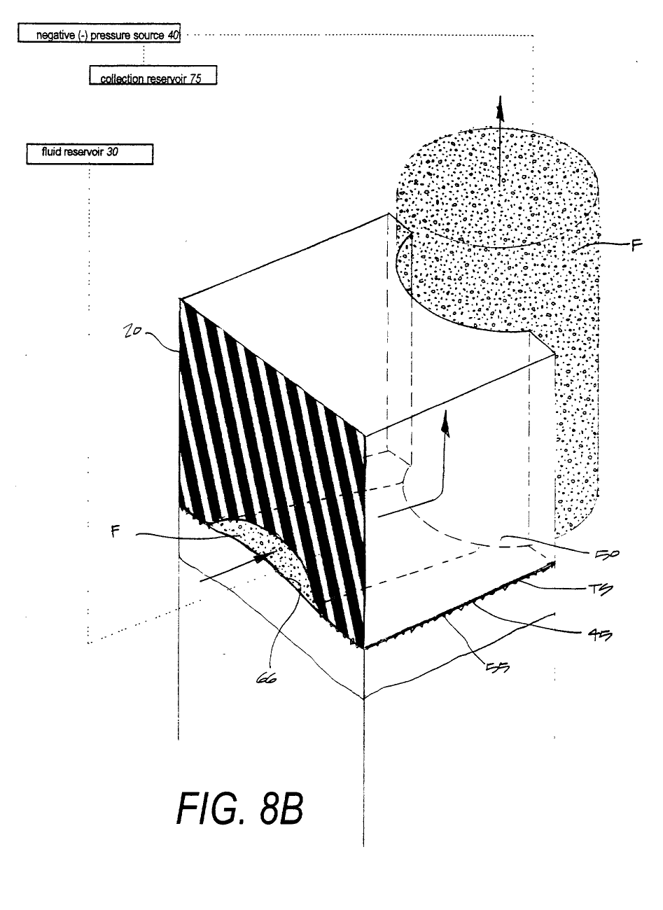

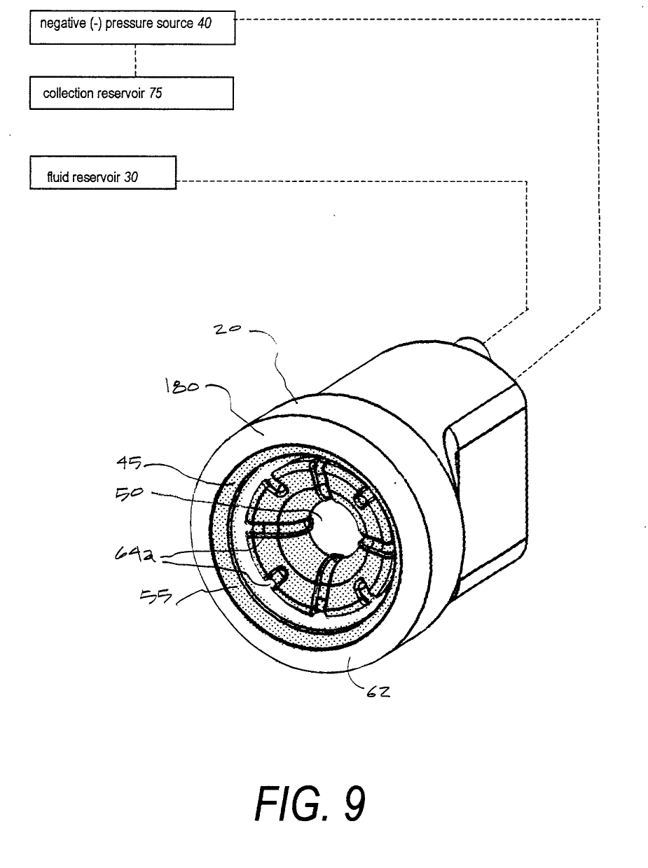

As can be seen in FIGS. 3-4, in this embodiment, the fluid inflow apertures 54 are located in one or more generally circumferential recessed portions or grooves 60a-60n in the skin interface 45 generally around opening 50. As can be understood easily, the negative pressure source 40 will draw fluid F into the concave form 48 of the working end when a perimeter 62 of the working end is pressed against a skin surface. It is desirable to have a suitable flow of fluid F generally across the opposing sides of the skin interface and into opening 50. It has been found that recesses or grooves 60a-60n of a selected dimension desirably maintain a ready amount of fluid F therein as the skin interface is moved over the skin. FIG. 6 shows a slightly different embodiment configured with two recesses or grooves 60a and 60b. FIG. 7 shows another slightly different embodiment with a plurality of inflow apertures 63a about a perimeter 62 of the working end without any substantial recesses or grooves but the apertures widening at their open distal termination indicated at 63b. It should be appreciated that the working end may be configured with micro-porosities (not shown) about the skin interface to serve as fluid inflow apertures 54 and fall within the scope of the invention. The optional grooves 60a-60n shown in FIGS. 3-4 are deep enough (having dimension D₁ in FIGS. 4 & 8A) so that the skin surface 66 can not be drawn entirely into the recess thus allowing the recess to be maintained with fluid therein during a treatment. It also has been found that generally radial recessed portions 64a-64n are useful for directing the flow of fluid F toward opening 50. The radial recessed portions or grooves 64a-64n have a depth D₂ shown in FIGS. 8A-8B that is generally shallower than the depth of grooves 60a-60n. By the term radial, it is meant that the recessed portions may extend directly toward opening 50 or at an angle relative to opening 50 and collectively, some of such grooves will generally be angled relative to the direction of translation of the working As shown in FIG. 8B, it is believed that the skin surface 66 will be slightly pulled into a groove 64a as the skin interface moves across skin and the fluid F within and about the groove will assist in removing skin detritus SD from the treatment site and be aspirated into central opening 50. The radial recessed portions or grooves 64a-64n may extend partly toward to central recess and opening 50 as shown in FIG. 3 or entirely to the central opening as shown in the alternative working end embodiment of FIG. 9.

Referring again to FIG. 4, it can be seen that working end 20 is detachable from body 42 by means of a male and female fitting. Working end 20 has a female or recessed portion 67 that sealably mates with the projecting portion 68 of body portion 42. Since working end 20 is of resilient material, the wall portion 69 of the working end 20 can be stretched and lip portion 72a locks into annular groove 73 of the body 42 to form a fluid-tight seal. The opening 50 of the working end 20 is then in alignment with interior passageway 51 of body 42. Likewise, the apertures 54 transition into an system of interior fluid flow channels 74a in the working end 20 that align with similar channels 74b in body 42 that communicate with fluid reservoir 30.

In the exemplary system, the aspiration source 40 thus has multiple simultaneous functions: (i) to draw the skin surface into the concave form 48 of working end 20 and more particularly against the skin interface 45 and diamond abrasive architecture to perform the method of removing skin surface layers; (ii) to draw a fluid F across the skin interface 45 diamond abrasive architecture 55 to remove and clean skin debris from the skin interface; and (iii) to further aspirate the skin debris and fluid volume F to a remote collection reservoir 75. Besides these functions, it has been found that patients find the fluid F to have a desirable cooling and hydrating effect (when compared to prior art high-velocity air-driven particle skin abrasion methods). The aspiration source or negative (−) pressurization source 40 may be any suitable vacuum source known in the art. The reservoir 30 is vented as is known in the art. Between the aspiration source 40 and the remote collection reservoir 75 is a filter 76 subsystem that is known in the art for collecting aspirated skin debris and fluid. The collection reservoir 75 and filter 76 are preferably of inexpensive plastic and other materials that are disposable.

The aspiration source 40 is preferably provided with a controller 80 and adjustable valve means 82 for adjusting the pressure level setting to any suitable range. The system operator will learn from experience how to balance the pressure level to attain the desired level of suction against the patient's skin. A trigger or switch component 78 is provided as a foot-switch (FIG. 2A) but any suitable finger switch in body 18 also may be used.

2. Practice of the Method of the Invention. Turning again to FIGS. 8A-8B, a sectional view of working end 20 shows the technique of the present invention in abrasive removal of skin surface layers. FIG. 8A shows the working end 20 after actuation of the negative (−) pressure source 40 with the skin surface 66 initially being drawn into the concave form 48 of the working end. The operating negative pressures may be in any suitable range that is determined by investigation. The flexibility of the resilient material of the working end allows the perimeter 62 of the working end to flex slightly to conform to the skin surface. It has been found by experimentation that optimal pressure levels vary greatly depending on (i) the type of skin targeted for treatment, (ii) the dimensions across the working end, and (iii) the dimensions of opening 50.

Next, the operator moves the skin interface 45 across a treatment site TS which is a path on the patient's skin while still actuating the trigger 78 thereby maintaining the negative pressure environment in the concavity 48 and opening 50. The sideways or generally lateral movement of the skin interface 45 allows the diamond architecture 55 to abrade the surface layers. Referring to FIG. 8B, the shallow radial groove 64a generally has a flow of fluid F (in the direction of arrows) therethrough which carries skin debris SD and fluid F to the opening 50 for aspiration to the collection reservoir 75. The translation of the skin interface 45 over the treatment site TS allows an abrasion and removal of the skin surface in a controllable manner.

It has been discovered that patients find the skin surface removal techniques disclosed herein to be substantially painless for limited depth surface removal. It has further been found that deeper skin surface removal procedures with the resilient working end 20 is substantially pain-free, particularly when compared to prior at skin removal methods for a similar depth treatment. It is believed that an important novel aspect of the invention is the suspension of the diamond fragments 55 in a resilient substrate such as silicone. It is postulated that the slight movement or adjustment of individual diamond fragments 55 of the diamond abrasive architecture in the resilient substrate as the skin interface 45 is translated over skin allows the diamonds to float to a slight extent and cut the skin surface in an atraumatic manner.

The negative pressure environment within the working end causes the fluid F and skin debris SD to be entrained in an air volume to be drawn through passageway 51 to the collection reservoir 75. After translating the working end over a treatment site, the operator may release trigger 78 to easily lift the working end from the patient's skin or simply reverse the movement of the device. The treated path can be easily seen and the operator then can remove skin layers in another slightly overlapping or adjacent path by repeating the above steps until surface removal is completed over the targeted treatment area. Following a treatment, of preferably a series of treatments over time, new skin surface layers including increased collagen aggregation in the papillary dermis will occur to provide a rejuvenated skin texture.

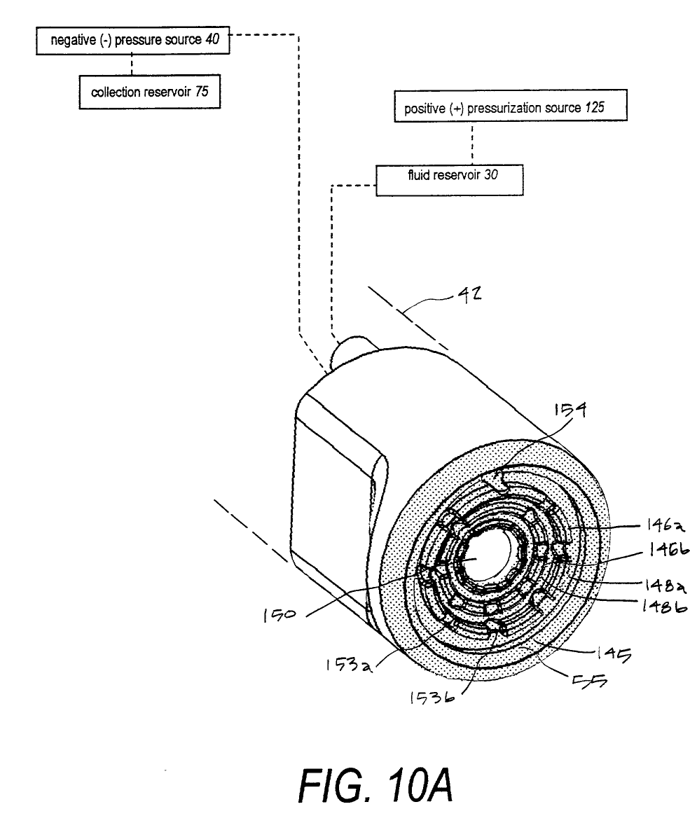

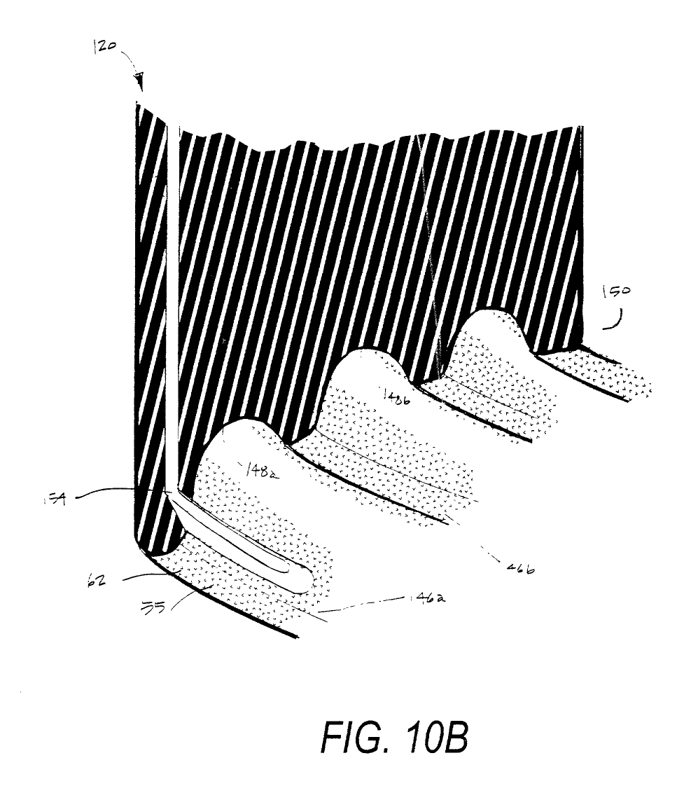

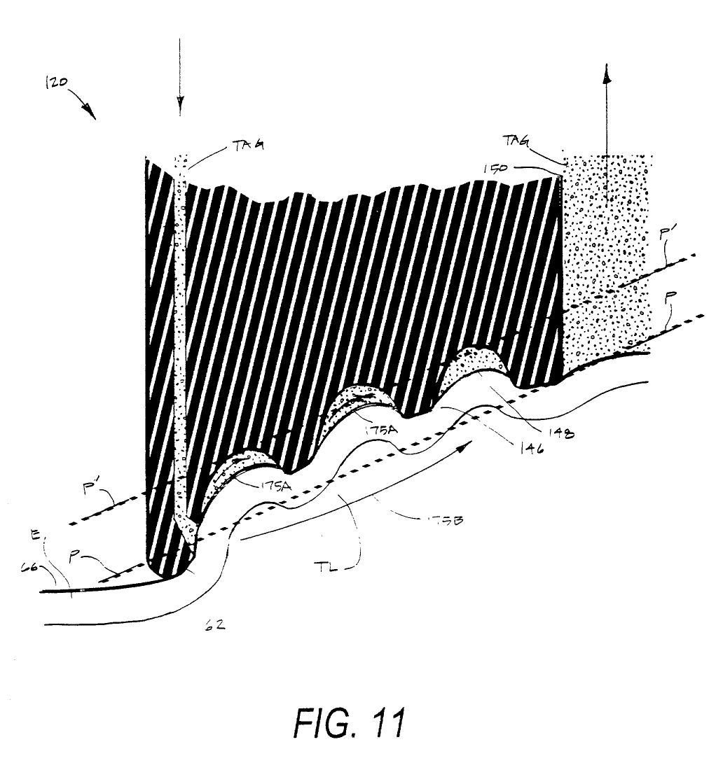

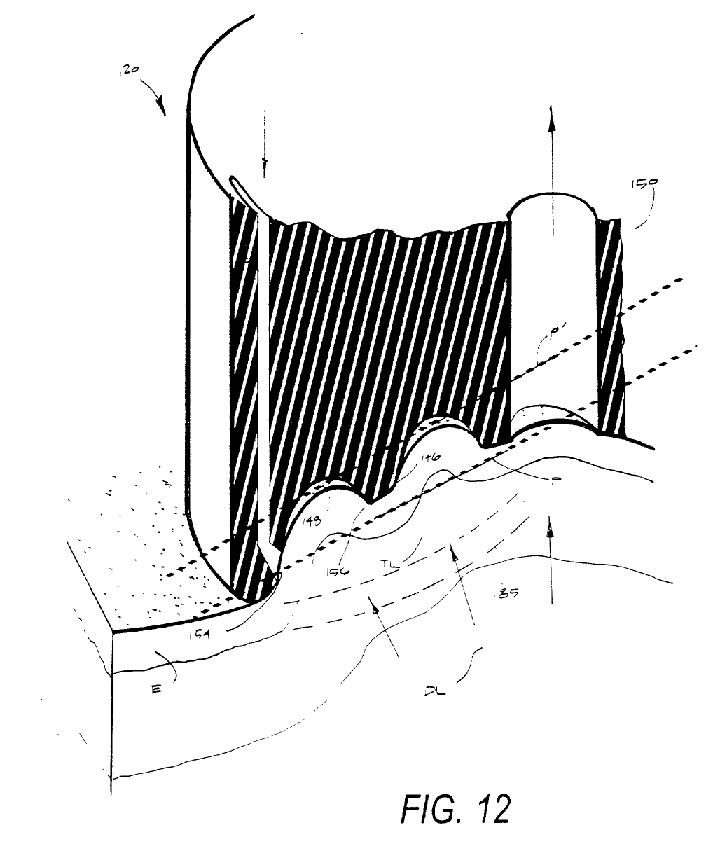

3. Type “B” Skin Treatment System. FIGS. 10A-10B and FIG. 11 illustrate an exemplary instrument system 105 and method of use that is similar to the previously described Type “A” system and is used to abrade skin surface layers. However, this Type “B” system is adapted to perform the additional inventive method of ultra-rapid subsurface delivery of a fluid therapeutic agent, either independently, or in combination with skin surface removal methods described above. The rapid subsurface delivery of therapeutic agents is adapted to enhance neocollagenesis, or otherwise deliver pharmacological agents to treat acne, or any other skin condition. Elements of the new Type “B” system that are functionally similar to elements of the previous embodiment have the same reference numerals+100; elements of the Type “B” system that are identical to those previously described have the previous reference numeral.

Referring now to FIG. 10B, a sectional view a Type “B” working end 120 is shown which is adapted for rapid subsurface delivery of a therapeutic agent TAG to a targeted layer indicated at TL. The working end is adapted to be fitted to a body 42 as described previously. In this embodiment, the fluid source 30 carries the therapeutic agent TAG and the source preferably is a disposable cartridge as described previously but also may be as remote source connected to body portion 42 by a flexible tube. Preferably, the therapeutic agent TAG is delivered about the skin surface 66 and rapidly beneath the skin surface by the negative (−) pressurization system 40 as described above. It should be appreciated that a positive (+) pressurization source 125 may be provided to move the therapeutic fluid from the storage reservoir to the working end 120 and fall within the scope of the invention, particularly when the reservoir in not built in to the handle.

Of particular interest, FIGS. 10A-10B show that the skin interface 145 of the therapeutic agent delivery system defines a plurality of spaced-apart projecting annular ridge elements 146a-146n with groove (recessed) portions 148a-148n therebetween that comprise a treatment region of the skin interface. These ridge elements 146a-146n and groove portions 148a-148n are generally annular or circumferential in relation to at least one media outflow port 150 that operates as described previously (i) to suction the skin interface against the patient's skin and (ii) to draw the therapeutic agent TAG from its storage reservoir. In this embodiment, a plurality of media inflow ports or apertures 154 for delivering the therapeutic agent TAG are configured about a perimeter 62 of the skin interface 145. In other words, the skin interface 145 defines an intermediate undulating region indicated at 156 comprising a plurality of ridge and groove elements that is intermediate to the arrangement of inflow apertures 154 and the outflow aperture 150 that define a distal plane P across the working end. Of particular importance, a proximalmost part of the recessed portions 148a-148n lies in a proximal plane P′ that is substantially proximal to the distal plane P that also carries the distalmost terminations of the media inflow and outflow ports. The number of ridge elements 146a-146n may be from about 2 to 20 depending on the overall size of the skin interface. Other shallow crossing grooves 153a-153n may be provided as described previously to direct fluid flow toward opening 150 while cleaning the diamond abrasive architecture of the skin interface (cf. FIG. 9). As can be seen in FIG. 10B, the diamond abrasive architecture 55 is carried about at least the surface of the ridge elements 146a-146n and portions of the groove elements 148a-148n (i) to suction the skin interface against the patient's skin and (ii) to draw the therapeutic agent TAG from its storage reservoir. Optionally, the abrasive architecture may be carried throughout the ridge and groove elements.

FIG. 11 shows a manner of practicing a method of the invention in delivering a therapeutic agent TAG to a targeted layer TL that is beneath skin surface 66 and epidermis E. The method includes actuating the negative pressure source 40 to (i) suction the skin interface 145 against the treatment site TS and (ii) further begin to draw the therapeutic agent TAG from storage reservoir 30 and through inflow apertures 154 toward the negative pressure in outflow opening 150. As can be seen in FIG. 11, a substantial negative pressure is applied to the skin interface to pull the skin surface 66 substantially into each groove element 148a-148n. Still referring to FIG. 11, it can be seen that there are basically two directions of travel that the therapeutic fluid agent TAG may traverse relative to intermediate region 156 of interface 145 from the outflow apertures 154 to the inflow aperture 150, which are indicated by arrows 175A and 175B. A first direction of fluid flow 175A is generally about and across skin interface 145 and over the skin surface 66 which has the advantage (described previously) of cleaning and scouring the abrasive architecture 55 and carrying skin detritus to opening 150. Of particular interest, it has been found that the substantial removal of the epidermal layer E by abrasive cutting allows for very rapid penetration of fluid into skin layers beneath the epidermis E, for example the targeted subsurface layers indicated at TL in FIG. 11. In other words, as shown in FIG. 11, a portion of the fluid agent TAG moves in the direction of least resistance indicated by arrow 175B toward opening 150 thereby delivering the therapeutic agent directly through the denuded skin surface 66 to the targeted subsurface layer TL. Without wishing to be limited to a particular theory that explains the improved method of subsurface fluid agent delivery, it is believed that the negative (−) pressurization source 40 creates a negative pressure condition within the interior of the patient's body about the targeted layer TL and within the tissue region captured in the recessed or groove elements 148a-148n to thereby draw the fluid agent TAG into the interior of the body after the epidermis is abraded. In this method of the invention, the therapeutic agent may be any suitable drug that is diluted into a fluid, for example a pharmacological agent in an acne treatment, an antibiotic, an anti-inflammatory agent, a steroid, an anesthetic, growth factors, or any other suitable agents known to stimulate the body's wound healing response to accelerate neocollagenesis in the targeted subsurface tissue layers.

Referring back to FIGS. 9-10, it has been found that the working end 120 preferably is surrounded by a perimeter member 180 of a lubricious material such as teflon. Additionally, the skin interface preferably is of a rigid material with abrasives affixed thereto or embedded therein. The method of delivering a therapeutic agent TAG to subsurface layers requires substantial negative pressures, and therefore the lubricious member 180 allows the working end to more easily glide over the patient's skin.

The Type “B” instrument of FIG. 10B and the method of FIG. 11 is adapted to perform another different method of the present invention for inducing neocollagenesis in a patient's skin. As described above, it has been found that inducing the body's wound healing response by epidermal removal is a critical component of a method of inducing neocollagenesis therein, as well as enhancing neocollagenesis by delivery of certain therapeutic agents. It is postulated the method of the invention provides an additional important element in enhancing and sustaining neocollagenesis to alter skin architecture. As shown in FIG. 12, the skin interface may have slightly deeper recessed portions 148a-148n to enhance capture of the skin surface layers within and by the intermediate region 156 between the first arrangement of media inflow ports or apertures 154 and cooperating second outflow port arrangement 150 with planes P and P′ as described above. Then, by developing a substantial negative pressure gradient (indicated at 185) in the targeted subsurface layers TL as well as in deeper layers DL, a significant amount of extracellular fluids EF will migrate or be drawn anteriorly from deeper (less anterior) layers of the dermis indicated at DL. The arrows in FIG. 12 illustrate the rapid migration of such extracellular fluids EF to the targeted layer Th and generally the upper layers (more anterior layers) of the dermis. It is believed that these extracellular fluids, which contain various growth factors etc., are important in enhancing and sustaining the level of neocollagenesis to alter the skin architecture and to increase collagen aggregates. It is believed that the method of the present invention by capturing the anteriormost skin layers in the recessed structures 148a-148n intermediate to cooperating first and second media inflow and outflow ports 154 and 150 can optimally cause a negative pressure condition in subsurface layers in the interior of the body in an atraumatic manner to thus cause and enhance neocollagenesis.

Specific features of the invention may be shown in some figures and not in others, and this is for convenience only and any feature may be combined with another in accordance with the invention. While the principles of the invention have been made clear in the exemplary embodiments, it will be obvious to those skilled in the art that modifications of the structure, arrangement, proportions, elements, and materials may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

What is claimed is:

1. A system for treating a patient's skin and/or for inducing neocollagenesis in skin, comprising:
(a) an instrument body with a working end that defines a skin interface for engaging a skin surface;
(b) an arrangement of media inflow and outflow port arrangements about said skin interface and defining a treatment region therebetween, wherein the outflow port arrangement communicates with a negative pressurization source and the inflow port arrangement communicates with a fluid reservoir; and
(c) wherein said treatment region carries at least one groove.

2. The system of claim 1 wherein the skin interface carries a plurality of annular grooves.

3. The system of claim 2 the grooves surround the media outflow port arrangement.

4. The system of claim 1 wherein at least a portion of the skin interface carries an abrasive surface architecture.

5. The system of claim 1 wherein the skin interface carries a plurality of media inflow ports about a periphery thereof.

6. The system of claim 1 wherein the proximalmost surface of the at least one groove of said treatment region defines a first proximal plane and the distalmost surface of the at least one groove defines a second proximal plane, and wherein the media inflow and outflow port arrangements are located distal to the first plane.

7. The system of claim 6 wherein the media outflow port arrangement is located substantially about the second plane.

8. The system of claim 6 wherein the media inflow port arrangement is located substantially about the second plane.

9. A method for treating a patient's skin, comprising:
(a) providing an instrument with a skin interface with a media inflow and media outflow port arrangements that defines a treatment region between the media inflow and outflow ports, wherein the treatment region carries at least one groove;
(b) actuating a negative pressurization source in fluid communication with the media outflow port arrangement thereby suctioning skin in contact with the skin interface;
(c) translating said skin interface across a treatment site; and
(d) wherein contemporaneous with steps (b) and (c), the negative pressurization source suctions a therapeutic fluid through the media inflow port arrangement and delivers the therapeutic fluid to subsurface tissue layers engaged by the skin interface.

10. The method of claim 9 wherein the subsurface delivery of the therapeutic fluid causes neocollagenesis in the patient's skin.

11. The method of claim 9 wherein said neocollagenesis in the patient's skin reduces wrinkles.

12. The method of claim 9 wherein the therapeutic fluid is selected from the class consisting of antibiotic agents, anti-inflammatory agents, anesthetic agents and growth factors.

13. The method of claim 9 wherein in steps (a) through (d) are repeated in periodic treatments over a treatment site.

14. The method of claim 9 wherein the skin interface carries an abrasive structure and the therapeutic fluid partly flows across the skin interface thereby removing skin debris.

15. A method for treating a patient's skin, comprising:
(a) providing an instrument with a skin interface with a media inflow and media outflow port arrangements that defines a treatment region between the media inflow and outflow ports, wherein the treatment region carries at least one groove;
(b) actuating a negative pressurization source in fluid communication with the media outflow port arrangement thereby suctioning skin in contact with the skin interface;
(c) translating said skin interface across a treatment site; and
(d) wherein steps (c) and (d) cause a negative pressure level in subsurface dermal layers thereby causing extracellular body fluids to migrate to more anterior layers from less anterior layers to cause neocollagenesis in the less anterior layers.
